(19) United States
(12) Patent Application Publication
(10) Pub. No.: US 2024/0000867 A1
(43) Pub. Date: Jan. 4, 2024

(54) SYNBIOTIC COMPOSITION

(71) Applicant: SYNBIOTICS AB, Lomma (SE)

(72) Inventors: Ulla K SVENSSON, Lund (SE); Catharina LAVEBRATT, Sollentuna (SE); Joëlle RÜEGG, Stockholm (SE); Martin SCHALLING, Stockholm (SE)

(73) Assignee: SYNBIOTICS AB, Lomma (SE)

(21) Appl. No.: 18/252,825

(22) PCT Filed: Nov. 12, 2021
(86) PCT No.: PCT/SE2021/051138

§ 371 Date: May 12, 2023

(30) Foreign Application Priority Data
Nov. 12, 2020 (SE) 2051327-1

(51) Int. Cl. A61K 35/745; A61K 35/747; A61K 31/702; A61K 31/718; A61K 31/732; A61K 31/733; A61P 1/00; A61P 29/00; A61P 31/14

(52) U.S. Cl. CPC A61K 35/745; A61K 35/747; A61K 31/702; A61K 31/718; A61K 31/732; A61K 31/733; A61P 1/00; A61P 29/00; A61P 31/14; C12R 2001/225

(57) ABSTRACT

The present application relates to a synbiotic composition for use in the prevention, amelioration, treatment, or reductions of symptoms in an individual suffering from Inflammatory Bowel Disease (IBD) or from an infection with a respiratory virus or from long-term effects after an infection with a respiratory virus, wherein the synbiotic composition comprises at least two bacterial strains chosen from the group consisting of Lactobacillus plantarum, Lactobacillus paracasei, Pediococcus pentosaceus, Leuconostoc mesenteroides, and Bifidobacterium breve; and at least one dietary fibers chosen from the group consisting of inulin, pectin, beta-glucan, resistant starch, a galacto-oligosaccharide, an isomalto-oligosaccharide and rice fibers.

### TECHNICAL FIELD

The present disclosure relates to synbiotic composition, comprising probiotic bacterial strains and prebiotic fibres or oligosaccharides, for use in the prevention, amelioration, treatment, or reductions of symptoms in an individual suffering from Inflammatory Bowel Disease (IBD) or from an infection with a respiratory virus or from long-term effects after an infection with a respiratory virus.

### BACKGROUND ART

During the last decades, the importance of the gut microbiota for wellbeing and health has been intensively studied. New knowledge has resulted in a suggestion to view the gut microbiota as a new organ. Molecular genetic methods have revolutionized the insight into the composition of the microbiota and microbiota analysis has made it possible to include studies of the microbiota as part of many human intervention studies. The gut with its microbiota has a key function to maintain immune homeostasis and approximately 70% of the immune system is estimated to be located in the gut.

Virus infections start with a virus entering the body and attaches to a virus receptor. After attachment, virus can enter into cells by different mechanisms and inside the cell the virus takes over the metabolism of the cell and multiplies. Some virus shares or partly shares receptors on the cell surface e.g. different upper tract respiratory viruses (Rhino-, Coxsackie- and Adenovirus). The immunoglobulin-like molecule CAR has been identified as a receptor for some types of Coxsackievirus and Adenovirus. Receptors for viruses usually have other functions besides being a receptor, e.g. ICAM (Intercellular adhesion molecule, also known as CD54 (Cluster of Differentiation 54)) occurs both as a soluble factor in serum and as a cell surface molecule. ICAM has been shown to be the receptor for Rhino- and Coxackievirus and is also important for the infection cycle of influenzavirus. The expression of ICAM has been shown to be enhanced in COVID-19 patients and patients with severe symptoms having the most enhanced expression. Another molecule with enhanced expression in COVID-19 patients is angiotensin and angiotensin converting enzyme (ACE). These proteins have been suggested to be important for attachment of SARS CoV-2-virus to the cell surface and have been observed to be more expressed in overweight persons and the elderly. This has been suggested to be one reason to that these two groups are at a high risk of getting a severe COVID-19 infection.

The incidence of upper respiratory tract infections is very high and some individuals suffer from several such infections per year. During the last year the coronavirus SARS-CoV2 has caused a pandemic of COVID-19, which in most individuals is a mild disease, but in some cases results in a life-threatening condition, requiring intensive care. In addition, COVID-19 can also result in prolonged illness and persistent symptoms, even in young adults and persons with no underlying medical conditions. There are very few effective treatments of upper respiratory tract infections and hardly no treatment for long-term effects of COVID-19.

An object of the present invention is to provide a composition for the prevention, amelioration, treatment, or reductions of symptoms in an individual suffering from an infection with a respiratory virus or from long-term effects after an infection with a respiratory virus.

### SUMMARY

According to a first aspect, the above and other objects of the invention are achieved, in full or at least in part by a composition as defined by claim 1. According to this claim, the above object is achieved by a synbiotic composition for use in the treatment, prevention, amelioration, or reductions of symptoms in an individual suffering from Inflammatory Bowel Disease (IBD) or from an infection with a respiratory virus or from long-term effects after an infection with a respiratory virus, wherein the synbiotic composition comprises at least two bacterial strains chosen from the group consisting of Lactobacillus plantarum, Lactobacillus paracasei, Pediococcus pentosaceus, Leuconostoc mesenteroides, and Bifidobacterium breve; and at least one dietary fiber chosen from the group consisting of inulin, pectin, beta-glucan, resistant starch, a galacto-oligosaccharide, an isomalto-oligosaccharide, and a rice fiber.

According to one embodiment, the Lactobacillus plantarum strain is Lactobacillus plantarum (LMG P-20606).

According to a second embodiment, the Lactobacillus paracasei strain is chosen from the group consisting of Lactobacillus paracasei (LMG P-17806), and Lactobacillus paracasei (LMG P-26118).

According to a further embodiment, the Pediococcus pentosaceus strain is Pediococcus pentosaceus (LMG P-20608); and/or the Leuconostoc mesenteroides strain is Leuconostoc mesenteroides (LMG P-20607); and/or the Bifidobacterium breve strain is Bifidobacterium breve (LMG-P-26117).

According to another embodiment, the composition comprises at least one Lactobacillus plantarum strain, preferably Lactobacillus plantarum (LMG P-20606), and at least one Lactobacillus paracasei strain, preferably Lactobacillus paracasei (LMG P-17806) or Lactobacillus paracasei (LMG P-26118).

According to one embodiment, the composition further comprises at least one Pediococcus pentosaceus strain, preferably Pediococcus pentosaceus (LMG P-20608).

According to another embodiment, the composition further comprises at least one Leuconostoc mesenteroides strain, preferably Leuconostoc mesenteroides (LMG P-20607).

According to a further embodiment, the composition further comprises at least one Bifidobacterium breve strain, preferably Bifidobacterium breve (LMG-P-26117).

According to yet another embodiment, the composition comprises Lactobacillus plantarum (LMG P-20606), Lactobacillus paracasei (LMG P-17806) or Lactobacillus paracasei (LMG P-26118), and Pediococcus pentosaceus (LMG P-20608).

According to one embodiment, the composition comprises inulin, pectin, beta-glucan and/or resistant starch.

According to another embodiment, the composition comprises resistant starch, a galacto-oligosaccharide, and/or an isomalto-oligosaccharide.

The composition may comprise a rice fiber.

The composition may comprises inulin, pectin, beta-glucan resistant starch and/or rice fibers.

According to one embodiment, the total amount of bacteria in one dose is 1×10⁶ to 1×10¹³, such as 5×10⁶ to 1×10¹³, such as 1×10⁷ to 1×10¹³, such as 5×10⁷ to 1×10¹³, such as 1×10⁸ to 1×10¹³, such as 5×10⁸ to 5×10¹², especially 1×10⁹ to 1×10¹², such as 5×10⁹ to 9×10¹¹, such as 1×10¹⁰ to 8×10¹¹, such as 5×10¹⁰ to 7×10¹¹, such as 1×10¹¹ to 6×10¹¹, such as 3×10¹¹ to 5×10¹¹, such as 4×10¹¹ CFUs (colony forming units).

According to a further embodiment, the total amount of fibers in one dose is between 0.1 to 20 g, such as 0.5 to 20 g, such as 1 to 20 g, such as 2 to 15 g, such as 2.5 to 12.5 g, such as 5 to 10 g, such as 6 to 8.5 g, such as 7.5 g.

The respiratory virus may be a virus that causes common cold, such as rhinovirus, adenovirus, or coronavirus.

According to one embodiment, the respiratory virus is chosen from the group consisting of adenovirus, rhinovirus, coxsackievirus, coronavirus, and influenzavirus.

The respiratory virus may be chosen from the group consisting of adenovirus, rhinovirus, coxsackievirus, and coronavirus.

The respiratory virus may be chosen from the group consisting of adenovirus, rhinovirus, and coxsackievirus.

The respiratory virus may be a coronavirus.

According to another embodiment, the coronavirus is chosen from the group consisting of SARS-CoV, MERS-CoV and SARS-CoV2.

According to yet another embodiment, the long-term effects after an infection with a respiratory virus are long-term effects after a COVID-19-infection.

According to another embodiment, the Inflammatory Bowel Disease (IBD) is ulcerative colitis or Crohn's disease.

According to one embodiment, the synbiotic composition is administered orally or rectally or by tube feeding.

According to another embodiment, the synbiotic composition is administered 1 to 3 times a day, 1 to 7 times a week.

According to a further embodiment, the synbiotic composition is in the form of a powder, a capsule, a tablet, a lozenge, a liquid, an emulsion, an enema, a suppository, or a tube feeding.

According to another embodiment, the synbiotic composition is a food supplement, a food product, a nutritional supplement, a natural remedy or a pharmaceutical product.

The food product may be a biscuit, a cracker, a bar, or a liquid, e.g. a shot.

The bacterial strains disclosed here in have previously been deposited with the Belgian Coordinated Collections of Micro-organisms (BCCM), Laboratorium voor Microbiologie Bacterisnverzameling (LMG), Universiteit Gent, K. L. Ledenganckstraat 35, 9000 Gent, Belgium (Web site: http://bccm.belspo.be).

Pediococcus pentosaceus (LMG P-20608), Lactobacillus plantarum (LMG P-20606), and Leuconostoc mesenteroides (LMG P-20607) were deposited on Jun. 19, 2001.

Lactobacillus paracasei (LMG P-17806) has been deposited with the Belgian Coordinated Collection of Microorganisms.

Lactobacillus paracasei (LMG P-26118) and Bifidobacterium breve (LMG P-26117) were deposited on Nov. 24, 2010.

Pediococcus pentosaceus (LMG P-20608), Lactobacillus plantarum (LMG P-20606), Lactobacillus paracasei (LMG P-17806) and Leuconostoc mesenteroides (LMG P-20607) have previously been published in EP 1 624 762 B1.

Lactobacillus paracasei (LMG P-26118) and Bifidobacterium breve (LMG P-26117) have previously been published in EP 2 672 980 B1.

Other objectives, features and advantages of the present disclosure will appear from the following detailed description, from the experimental data, as well as from the attached claims. It is noted that the disclosure relates to all possible combination of features.

Generally, all terms used in the claims are to be interpreted according to their ordinary meaning in the technical field, unless explicitly defined otherwise herein. All references to “a/an/the [component, means, step, etc.]” are to be interpreted openly as referring to at least one instance of said component, means, step, etc., unless explicitly stated otherwise. The steps of any method disclosed herein do not have to be performed in the exact order disclosed, unless explicitly stated.

As used herein, the term “comprising” and variations of that term are not intended to exclude other additives, components, integers or steps.

### BRIEF DESCRIPTION OF DRAWINGS

FIG. 1a shows a schematic overview of a study timetable, its design and animals' randomization.

FIG. 1b. shows histological evaluation of colon structure and colitis severity. Intact=Control group of intact healthy mice (n=3); #37, #56, #47, #50: animals receiving 1% DSS in drinking water and treated daily with respective products (Products 1-4) (n=3); MEZ: animals receiving 1% DSS in drinking water and treated daily with Mesalamine suspension in olive oil (100 mg/kg body weight) (n=3); Placebo: group receiving 1% DSS in drinking water and daily treated with water gavage as a vehicle (n=3). Data were analysed using one-way ANOVA test corrected for multiple comparisons and are presented as mean±SD. Small letters describes significant differences when p<0.05.

FIGS. 1c-1f show the change in level over time for the 4 markers with statistically significant changes over time for all 4 products, here exemplified by the group treated with Product 3 (corresponding to Composition No. 47). The marker changes in the group treated with clinical pharmacotherapy for IBD, Mesalamine, is also shown.

FIG. 2a shows the responses from respondents who have used a composition according to the present disclosure for different time periods to the question “Do you feel that the synbiotic composition helped you with problems with colds/seasonal flu?”

### DETAILED DESCRIPTION

The gut with its microbiota has a key function to maintain immune homeostasis and approximately 70% of the immune system is estimated to be located in the gut.

Probiotics are defined as “a live microorganisms which, when administered in adequate amounts, confer a health benefit on the host” (FAO/WHO 2002).

Fibres are an essential part of our diet. Fibres can be divided into different groups and the group called soluble fibre are important for the gut microbiota. A group of the soluble fibres and oligosaccharides are called prebiotics.

Prebiotics are defined as “a substrate that is selectively utilized by host microorganisms conferring a health benefit” (International Scientific Association for Probiotics and Prebiotics 2017). When prebiotics are fermented in the gut, short chain fatty acids can be produced. Examples of the short chain fatty acids are acetic acid, butyric acid, propionic acid, and formic acid. It is known that short chain fay acids influence health, e.g. butyric acid nourishes the gut mucosa and propionic acid influences fat metabolism.

Synbiotics is defined as “an ingredient or dietary supplement combining probiotics and prebiotics for a synergy between the compounds”.

The combination of strains presented herein and used in the studies and experiments were carefully selected to be optimal combination of strains for efficacy including bacterial survival and favourable influence on each other.

Different combinations of bacterial strains and fibers may be used in a composition according to the present disclosure. Different combinations of bacterial strains and different combinations of fibers are described below. Specific combinations of bacterial strains and fibers are shown in Table 1. However, the following is not to be interpreted as excluding other combinations of bacterial strains and fibers.

Throughout the present disclosure, specific bacterial strains are identified by their deposition number. Thus, “Lactobacillus plantarum (LMG P-20606)” can be denoted as “Lactobacillus plantarum (deposit no: LMG P-20606)” or “Lactobacillus plantarum LMG P-20606”, all meaning a strain of Lactobacillus plantarum having the deposition number “LMG P-20606”.

Compositions according to the present disclosure comprise at least two bacterial strains chosen from the group consisting of Lactobacillus plantarum, Lactobacillus paracasei, Pediococcus pentosaceus, Leuconostoc mesenteroides, Bifidobacterium breve.

The composition may comprise one Lactobacillus plantarum strain and one Lactobacillus paracasei strain.

The composition may comprise Lactobacillus plantarum (LMG P-20606) and Lactobacillus paracasei (LMG P-17806).

The composition may comprise Lactobacillus plantarum (LMG P-20606) and Lactobacillus paracasei (LMG P-26118).

The composition may comprise one Lactobacillus plantarum strain, one Lactobacillus paracasei strain and one Pediococcus pentosaceus strain.

The composition may comprise Lactobacillus plantarum (LMG P-20606), Lactobacillus paracasei (LMG P-17806), and Pediococcus pentosaceus (LMG P-20608).

The composition may comprise Lactobacillus plantarum (LMG P-20606), Lactobacillus paracasei (LMG P-26118), and Pediococcus pentosaceus (LMG P-20608).

The composition may comprise one Lactobacillus plantarum strain, one Lactobacillus paracasei strain, one Pediococcus pentosaceus strain, and one Leuconostoc mesenteroides strain.

The composition may comprise Lactobacillus plantarum (LMG P-20606), Lactobacillus paracasei (LMG P-17806), Pediococcus pentosaceus (LMG P-20608) and Leuconostoc mesenteroides (LMG P-20607).

The composition may comprise Lactobacillus plantarum (LMG P-20606), Lactobacillus paracasei (LMG P-26118), Pediococcus pentosaceus (LMG P-20608) and Leuconostoc mesenteroides (LMG P-20607).

The composition may comprise one Lactobacillus plantarum strain, one Lactobacillus paracasei strain, one Pediococcus pentosaceus strain, and one Bifidobacterium breve strain.

The composition may comprise Lactobacillus plantarum (LMG P-20606), Lactobacillus paracasei (LMG P-17806), Pediococcus pentosaceus (LMG P-20608) and Bifidobacterium breve (LMG-P-26117).

The composition may comprise Lactobacillus plantarum (LMG P-20606), Lactobacillus paracasei (LMG P-26118), Pediococcus pentosaceus (LMG P-20608) and Bifidobacterium breve (LMG-P-26117).

The composition may comprise one Lactobacillus plantarum strain, one Lactobacillus paracasei strain, one Pediococcus pentosaceus strain, one Leuconostoc mesenteroides strain and one Bifidobacterium breve strain.

The composition may comprise Lactobacillus plantarum (LMG P-20606), Lactobacillus paracasei (LMG P-17806), Pediococcus pentosaceus (LMG P-20608), Leuconostoc mesenteroides (LMG P-20607) and Bifidobacterium breve (LMG-P-26117).

The composition may comprise Lactobacillus plantarum (LMG P-20606), Lactobacillus paracasei (LMG P-26118), Pediococcus pentosaceus (LMG P-20608), Leuconostoc mesenteroides (LMG P-20607) and Bifidobacterium breve (LMG-P-26117).

The composition may comprise Lactobacillus plantarum (LMG P-20606), Lactobacillus paracasei (LMG P-17806), Pediococcus pentosaceus (LMG P-20608). Preferably, the composition also comprises Leuconostoc. mesenteroides (LMG P-20607) and/or Bifidobacterium breve (LMG-P-26117).

The composition may comprise Lactobacillus plantarum (LMG P-20606), Lactobacillus paracasei (LMG P-26118), Pediococcus pentosaceus (LMG P-20608). Preferably, the composition also comprises Leuconostoc. mesenteroides (LMG P-20607) and/or Bifidobacterium breve (LMG-P-26117).

The composition may comprise Lactobacillus plantarum (LMG P-20606); Lactobacillus paracasei (LMG P-17806); Pediococcus pentosacceus (LMG P-20608); Leuconostoc mesenterioides (LMG P-20607); and Bifidobacterium breve (LMG P-26117).

The composition may comprise Lactobacillus plantarum (LMG P-20606); Lactobacillus paracasei (LMG P-26118); Pediococcus pentosacceus (LMG P-20608); Leuconostoc mesenterioides (LMG P-20607); and Bifidobacterium breve (LMG P-26117).

The composition may comprise Lactobacillus plantarum (LMG P-20606) and Lactobacillus paracasei (LMG P-17806).

The composition may comprise Lactobacillus plantarum (LMG P-20606) and Bifidobacterium breve (LMG P-26117).

Compositions according to the present disclosure comprise at least one fiber chosen from the group consisting of inulin, pectin, beta-glucan, resistant starch, a galacto-oligosaccharide, an isomalto-oligosaccharide, and a rice fiber.

The composition may comprise resistant starch.

The composition may comprise inulin, pectin, beta-glucan and resistant starch.

The composition may comprise inulin, pectin, beta-glucan, rice fiber and resistant starch.

The composition may comprise resistant starch and a galacto-oligo-saccharide.

The composition may comprise resistant starch and an isomalto-oligo-saccharide.

The composition may comprise resistant starch, galacto-oligosaccharide and isomalto-oligosaccharide.

A composition according to the present invention may be administered one to three times a day.

Some examples of compositions according to the present disclosure are shown in Table 1 below.

A composition according to the present invention may be administered one to seven days a week.

Preferably, a composition according to the present invention is administered once a day as a single dose.

The total amount of bacteria in one dose of a composition according to the present disclosure is between 1×10⁶ to 1×10¹³, such as 5×10⁶ to 1×10¹³, such as 1×10⁷ to 1×10¹³, such as 5×10⁷ to 1×10¹³, such as 1×10⁸ to 1×10¹³, such as 5×10⁸ to 5×10¹², especially 1×10⁹ to 1×10¹², such as 5×10⁹ to 9×10¹¹, such as 1×10¹⁰ to 8×10¹¹, such as 5×10¹⁰ to 7×10¹¹, such as 1×10¹¹ to 6×10¹¹, such as 3×10¹¹ to 5×10¹¹, such as 4×10¹¹ CFUs (colony forming units).

The total amount of fibers in one dose of a composition according to the present disclosure is between 0.1 to 20 g, such as 0.5 to 20 g, such as 1 to 20 g, such as 2 to 15 g, such as 2.5 to 12.5 g, such as 5 to 10 g, such as 6 to 8.5 g, such as 7.5 g.

A composition according to the present disclosure may be in the form of a dry powder intended to be mixed with a liquid, preferably water, before intake.

A composition according to the present disclosure may be in the form of a capsule, a tablet, a lozenge, a liquid, an emulsion, an enema, a suppository, or a tube feeding.

TABLE 1
Compositions according to the present disclosure. The total
amount of bacteria in one dose is between 1 × 10⁶ to
1 × 10¹³, such as 5 × 10⁶ to 1 × 10¹³, such as 1 × 10⁷
to 1 × 10¹³, such as 5 × 10⁷ to 1 × 10¹³, such as 1 × 10⁸ to 1 × 10¹³,
such as 5 × 10⁸ to 5 × 10¹², especially 1 × 10⁹ to 1 × 10¹², such as 5 × 10⁹ to
9 × 10¹¹, such as 1 × 10¹⁰ to 8 × 10¹¹, such as 5 × 10¹⁰ to 7 × 10¹¹,
such as 1 × 10¹¹ to 6 × 10¹¹, such as 3 × 10¹¹ to 5 × 10¹¹, such as 4 × 10¹¹
CFUs (colony forming units). The total amount of fibers
in one dose is between 0.1 to 20 g, such as 0.5 to 20 g,
such as 1 to 20 g, such as 2 to 15 g, such as 2.5 to 12.5 g,
such as 5 to 10 g, such as 6 to 8.5 g, such as 7.5 g. In
addition to the fibers listed below, rice fiber may also
be present. Combinations No. 1-57 also including rice
fibers are thus also possible.

| | Bacterial strains (at least two) | | | | | |
|---|---|---|---|---|---|---|
| Composition | L. plantarum | L. paracasei | L. paracasei | P. pentosaceus | L. mesenteroides | B. breve |
| No. | (LMG P-20606) | (LMG P-17806) | (LMG P-26118) | (LMG P-20608) | (LMG P-20607) | (LMG-P-26117) |
| 1 | x | x | | x | | |
| 2 | x | x | | x | | |
| 3 | x | x | | x | | |
| 4 | x | | x | x | | |
| 5 | x | | x | x | | |
| 6 | x | | x | x | | |
| 7 | x | x | | | x | |
| 8 | x | x | | | x | |
| 9 | x | x | | | x | |
| 10 | x | | x | | x | |
| 11 | x | | x | | x | |
| 12 | x | | x | | x | |
| 13 | x | x | | | | x |
| 14 | x | x | | | | x |
| 15 | x | x | | | | x |
| 16 | x | | x | | | x |
| 17 | x | | x | | | x |
| 18 | x | | x | | | x |
| 19 | x | x | | x | x | |
| 20 | x | x | | x | x | |
| 21 | x | x | | x | x | |
| 22 | x | | x | x | x | |
| 23 | x | | x | x | x | |
| 24 | x | | x | x | x | |
| 25 | x | x | | x | | x |
| 26 | x | x | | x | | x |
| 27 | x | x | | x | | x |
| 28 | x | | x | x | | x |
| 29 | x | | x | x | | x |
| 30 | x | | x | x | | x |
| 31 | x | x | | | x | x |
| 32 | x | x | | | x | x |
| 33 | x | x | | | x | x |
| 34 | x | | x | | x | x |
| 35 | x | | x | | x | x |
| 36 | x | | x | | x | x |
| 37 | x | x | | x | x | x |
| 38 | x | x | | x | x | x |
| 39 | x | x | | x | x | x |
| 40 | x | | x | x | x | x |
| 41 | x | | x | x | x | x |
| 42 | x | | x | x | x | x |
| 43 | x | x | | | | |
| 44 | x | x | | | | |
| 45 | x | x | | | | |
| 46 | x | | x | | | |
| 47 | x | | x | | | |
| 48 | x | | x | | | |
| 49 | x | | | x | | |
| 50 | x | | | x | | |
| 51 | x | | | x | | |
| 52 | x | | | | x | |
| 53 | x | | | | x | |
| 54 | x | | | | x | |
| 55 | x | | | | | x |
| 56 | x | | | | | x |
| 57 | x | | | | | x |

| | Fiber (at least one) | | | | | |
|---|---|---|---|---|---|---|
| Composition | | | beta- | resistant | galacto- | isomalto- |
| No. | inulin | pectin | glucan | starch | oligosaccharide | oligosaccharide |
| 1 | x | x | x | x | | |
| 2 | | | | x | | |
| 3 | | | | x | x | x |
| 4 | x | x | x | x | | |
| 5 | | | | x | | |
| 6 | | | | x | x | x |
| 7 | x | x | x | x | | |
| 8 | | | | x | | |
| 9 | | | | x | x | x |
| 10 | x | x | x | x | | |
| 11 | | | | x | | |
| 12 | | | | x | x | x |
| 13 | x | x | x | x | | |
| 14 | | | | x | | |
| 15 | | | | x | x | x |
| 16 | x | x | x | x | | |
| 17 | | | | x | | |
| 18 | | | | x | x | x |
| 19 | x | x | x | x | | |
| 20 | | | | x | | |
| 21 | | | | x | x | x |
| 22 | x | x | x | x | | |
| 23 | | | | x | | |
| 24 | | | | x | x | x |
| 25 | x | x | x | x | | |
| 26 | | | | x | | |
| 27 | | | | x | x | x |
| 28 | x | x | x | x | | |
| 29 | | | | x | | |
| 30 | | | | x | x | x |
| 31 | x | x | x | x | | |
| 32 | | | | x | | |
| 33 | | | | x | x | x |
| 34 | x | x | x | x | | |
| 35 | | | | x | | |
| 36 | | | | x | x | x |
| 37 | x | x | x | x | | |
| 38 | | | | x | | |
| 39 | | | | x | x | x |
| 40 | x | x | x | x | | |
| 41 | | | | x | | |
| 42 | | | | x | x | x |
| 43 | x | x | x | x | | |
| 44 | | | | x | | |
| 45 | | | | x | x | x |
| 46 | x | x | x | x | | |
| 47 | | | | x | | |
| 48 | | | | x | x | x |
| 49 | x | x | x | x | | |
| 50 | | | | x | | |
| 51 | | | | x | x | x |
| 52 | x | x | x | x | | |
| 53 | | | | x | | |
| 54 | | | | x | x | x |
| 55 | x | x | x | x | | |
| 56 | | | | x | | |
| 57 | | | | x | x | x |

Patients suffering from mild to moderate forms of adenovirus infection may be administered a composition according to the present disclosure in the form of a powder suspended in a liquid, preferably water, or in the form of a capsule, a tablet, a lozenge, a liquid, an emulsion, an enema, a suppository, or by tube feeding.

Patients suffering from severe forms of adenovirus infection may be administered a composition according to the present disclosure in the form of a tube feeding, an enema, or a suppository.

Patients suffering from mild to moderate forms of rhinovirus infection may be administered a composition according to the present disclosure in the form of a powder suspended in a liquid, preferably water, or in the form of a capsule, a tablet, a lozenge, a liquid, an emulsion, an enema, or a suppository, or by tube feeding.

Patients suffering from severe forms of rhinovirus infection may be administered a composition according to the present disclosure in the form of a tube feeding, an enema, or a suppository.

Patients suffering from mild to moderate forms of coxsackievirus infection may be administered a composition according to the present disclosure in the form of a powder suspended in a liquid, preferably water, or in the form of a capsule, a tablet, a lozenge, a liquid, an emulsion, an enema, or a suppository, or by tube feeding.

Patients suffering from severe forms of coxsackievirus infection may be administered a composition according to the present disclosure in the form of a tube feeding, an enema, or a suppository.

Patients suffering from mild to moderate forms of coronavirus infection may be administered a composition according to the present disclosure in the form of a powder suspended in a liquid, preferably water, or in the form of a capsule, a tablet, a lozenge, a liquid, an emulsion, an enema, or a suppository, or by tube feeding.

Patients suffering from severe forms of coronavirus infection may be administered a composition according to the present disclosure in the form of a tube feeding, an enema, or a suppository.

Patients suffering from mild to moderate forms of COVID-19 may be administered a composition according to the present disclosure in the form of a powder suspended in a liquid, preferably water, or in the form of a capsule, a tablet, a lozenge, a liquid, an emulsion, an enema, or a suppository, or by tube feeding.

Patients suffering from severe forms of COVID-19 may be administered a composition according to the present disclosure in the form of a tube feeding, an enema, or a suppository.

Patients suffering from long-term effects of COVID-19 may be administered a composition according to the present disclosure in the form of a powder suspended in a liquid, preferably water, or in the form of a capsule, a tablet, a lozenge, a liquid, an emulsion, an enema, or a suppository, or by tube feeding.

Several different viruses may cause respiratory infections. Symptoms of such infections include fever, cough, sore throat, headache, fatigue, difficulties to breathe and/or reduced lung volume. A virus infection starts with the virus entering into the body and attaches to a virus receptor on the surface of cells in the respiratory tract. After attachment, the virus can enter into cells by different mechanisms and inside the cell the virus takes over the metabolism of the cell and multiplies.

Adenovirus, rhinovirus and coxsackievirus all belong to the group of naked viruses, i.e. they are not surrounded by a membrane. They attach to a receptor through the proteins on their surface. Adenovirus have spikes called fibers on the surface, and the fibers attach to the receptors on the cell surface.

One composition that is specifically suitable for the treatment, amelioration or reduction of symptoms of an infection with adenovirus, rhinovirus and/or coxsackievirus comprises Lactobacillus plantarum (LMG P-20606), Lactobacillus paracasei (LMG P-17806), Pediococcus pentosaceus (LMG P-20608). Preferably, the composition also comprises Leuconostoc. mesenteroides (LMG P-20607) and/or Bifidobacterium breve (LMG-P-26117).

Another composition that is specifically suitable for the treatment, amelioration or reduction of symptoms of an infection with adenovirus, rhinovirus and/or coxsackievirus comprises Lactobacillus plantarum (LMG P-20606), Lactobacillus paracasei (LMG P-26118), Pediococcus pentosaceus (LMG P-20608). Preferably, the composition also comprises Leuconostoc mesenteroides (LMG P-20607) and/or Bifidobacterium breve (LMG-P-26117).

A further composition that is specifically suitable for the treatment, amelioration or reduction of symptoms of an infection with adenovirus, rhinovirus and/or coxsackievirus comprises Lactobacillus plantarum (LMG P-20606); Lactobacillus paracasei (LMG P-17806); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of an infection with adenovirus, rhinovirus and/or coxsackievirus comprises Lactobacillus plantarum (LMG P-20606); Lactobacillus paracasei (LMG P-26118); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of an infection with adenovirus, rhinovirus and/or coxsackievirus comprises Lactobacillus plantarum (LMG P-20606) and Lactobacillus paracasei (LMG P-17806).

A further composition that is specifically suitable for the treatment, amelioration or reduction of symptoms of an infection with adenovirus, rhinovirus and/or coxsackievirus comprises Lactobacillus plantarum (LMG P-20606) and Bifidobacterium breve (LMG P-26117).

Compositions that are specifically suitable for the treatment, amelioration or reduction of symptoms of an infection with adenovirus, rhinovirus and/or coxsackievirus are Composition Nos. 19, 22, 37, 40, 47, 50 and 56 in Table 1.

Coronavirus including SARS-CoV (severe acute respiratory syndrome coronavirus), MERS-CoV (Middle East respiratory syndrome coronavirus) and SARS CoV-2 (severe acute respiratory syndrome coronavirus 2) and influenza-virus belong to the enveloped viruses, i.e. they are all surrounded by a membrane originating from the plasma membrane of the host cell. Both coronavirus and influenzavirus have protein structures i.e. spikes in their membranes consisting of proteins that attach to the receptors during infection.

Common symptoms associated with Covid-19 caused by the coronavirus SARS-CoV2 are fever or chills, cough, sore throat, runny or stuffy nose, muscle or body aches, headaches, and fatigue.

One composition that is specifically suitable for the treatment, amelioration or reduction of symptoms of an infection with coronavirus comprises Lactobacillus plantarum (LMG P-20606), Lactobacillus paracasei (LMG P-17806), Pediococcus pentosaceus (LMG P-20608). Preferably, the composition also comprises Leuconostoc mesenteroides (LMG P-20607) and/or Bifidobacterium breve (LMG-P-26117).

Another composition that is specifically suitable for the treatment, amelioration or reduction of symptoms of an infection with coronavirus comprises Lactobacillus plantarum (LMG P-20606), Lactobacillus paracasei (LMG P-26118), Pediococcus pentosaceus (LMG P-20608). Preferably, the composition also comprises Leuconostoc mesenteroides (LMG P-20607) and/or Bifidobacterium breve (LMG-P-26117).

A further composition that is specifically suitable for the treatment, amelioration or reduction of symptoms of an infection with coronavirus comprises Lactobacillus plantarum (LMG P-20606); Lactobacillus paracasei (LMG P-17806); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of an infection with coronavirus comprises Lactobacillus plantarum (LMG P-20606); Lactobacillus paracasei (LMG P-26118); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of an infection with coronavirus comprises Lactobacillus plantarum (LMG P-20606) and Lactobacillus paracasei (LMG P-17806).

A further composition that is specifically suitable for the treatment, amelioration or reduction of symptoms of an infection with coronavirus comprises Lactobacillus plantarum (LMG P-20606) and Bifidobacterium breve (LMG P-26117).

Compositions that are specifically suitable for the treatment, amelioration or reduction of symptoms of an infection with coronavirus are Composition Nos. 19, 22, 37, 40, 47, 50 and 56 in Table 1.

For some people, COVID-19 can cause symptoms that last weeks or months after recovery from the initial infection with the SARS-CoV2-virus. This is referred to as post-COVID-19 syndrome, “long COVID” or long term effects after a COVID-19-infection. More than 200 different symptoms have been reported in patients suffering from post-COVID-19 syndrome. Common symptoms include e.g. extreme tiredness (fatigue), shortness of breath, chest pain or tightness, problems with memory and concentration (“brain fog”), difficulty sleeping (insomnia), heart palpitations, dizziness, joint pain, depression and anxiety, tinnitus, earaches, feeling sick, diarrhoea, stomach aches, loss of appetite, a high temperature, cough, headaches, sore throat, changes to sense of smell or taste, rashes. One or more of these symptoms may be treated, ameliorated or reduced according to the present disclosure.

Specifically, symptom(s) of post-COVID-19 syndrome that may be treated, ameliorated or reduced according to the present disclosure may be one or more of symptoms chosen from the group consisting of extreme tiredness (fatigue), problems with memory and concentration (“brain fog”), headaches, cough, heart palpitations, and changes to sense of smell or taste.

One composition that is specifically suitable for the treatment, amelioration or reduction of symptoms of long term effects after a COVID-19-infection comprises Lactobacillus plantarum (LMG P-20606), Lactobacillus paracasei (LMG P-17806), Pediococcus pentosaceus (LMG P-20608). Preferably, the composition also comprises Leuconostoc mesenteroides (LMG P-20607) and/or Bifidobacterium breve (LMG-P-26117).

Another composition that is specifically suitable for the treatment, amelioration or reduction of symptoms of long term effects after a COVID-19-infection comprises Lactobacillus plantarum (LMG P-20606), Lactobacillus paracasei (LMG P-26118), Pediococcus pentosaceus (LMG P-20608). Preferably, the composition also comprises Leuconostoc mesenteroides (LMG P-20607) and/or Bifidobacterium breve (LMG-P-26117).

A further composition that is specifically suitable for the treatment, amelioration or reduction of symptoms of long term effects after a COVID-19-infection comprises Lactobacillus plantarum (LMG P-20606); Lactobacillus paracasei (LMG P-17806); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of an infection with long term effects after a COVID-19-infection comprises Lactobacillus plantarum (LMG P-20606); Lactobacillus paracasei (LMG P-26118); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of long term effects after a COVID-19-infection comprises Lactobacillus plantarum (LMG P-20606) and Lactobacillus paracasei (LMG P-17806).

A further composition that is specifically suitable for the treatment, amelioration or reduction of symptoms of long term effects after a COVID-19-infection comprises Lactobacillus plantarum (LMG P-20606) and Bifidobacterium breve (LMG P-26117).

Compositions that are specifically suitable for the treatment, amelioration or reduction of symptoms of long term effects after a COVID-19-infection are Composition Nos. 19, 22, 37, 40, 47, 50 and 56 in Table 1.

Common symptoms associated with flue/seasonal flu caused by influenzavirus are fever or chills, cough, sore throat, runny or stuffy nose, muscle or body aches, headaches, and fatigue.

One composition that is specifically suitable for the treatment, amelioration or reduction of symptoms of an infection with influenzavirus comprises Lactobacillus plantarum (LMG P-20606), Lactobacillus paracasei (LMG P-17806), Pediococcus pentosaceus (LMG P-20608). Preferably, the composition also comprises Leuconostoc mesenteroides (LMG P-20607) and/or Bifidobacterium breve (LMG-P-26117).

Another composition that is specifically suitable for the treatment, amelioration or reduction of symptoms of an infection with influenzavirus comprises Lactobacillus plantarum (LMG P-20606), Lactobacillus paracasei (LMG P-26118), Pediococcus pentosaceus (LMG P-20608). Preferably, the composition also comprises Leuconostoc mesenteroides (LMG P-20607) and/or Bifidobacterium breve (LMG-P-26117).

A further composition that is specifically suitable for the treatment, amelioration or reduction of symptoms of an infection with influenzavirus comprises Lactobacillus plantarum (LMG P-20606); Lactobacillus paracasei (LMG P-17806); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of an infection with influenzavirus comprises Lactobacillus plantarum (LMG P-20606); Lactobacillus paracasei (LMG P-26118); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of an infection with influenzavirus comprises Lactobacillus plantarum (LMG P-20606) and Lactobacillus paracasei (LMG P-17806).

A further composition that is specifically suitable for the treatment, amelioration or reduction of symptoms of an infection with influenzavirus comprises Lactobacillus plantarum (LMG P-20606) and Bifidobacterium breve (LMG P-26117).

Compositions that are specifically suitable for the treatment, amelioration or reduction of symptoms of an infection with influenzavirus are Composition Nos. 19, 22, 37, 40, 47, 50 and 56 in Table 1.

Inflammatory bowel disease (IBD) is a term for two conditions (Crohn's disease and ulcerative colitis) that are characterized by chronic inflammation of the gastrointestinal (GI) tract. Prolonged inflammation results in damage to the GI tract. Some common symptoms are persistent diarrhea, abdominal pain, rectal bleeding/bloody stools, weight loss, and fatigue. One or more of these symptoms may be treated, ameliorated or reduced according to the present disclosure.

One composition that is specifically suitable for the treatment, amelioration or reduction of symptoms of IBD comprises Lactobacillus plantarum (LMG P-20606), Lactobacillus paracasei (LMG P-17806), Pediococcus pentosaceus (LMG P-20608). Preferably, the composition also comprises Leuconostoc mesenteroides (LMG P-20607) and/or Bifidobacterium breve (LMG-P-26117).

Another composition that is specifically suitable for the treatment, amelioration or reduction of symptoms of IBD comprises Lactobacillus plantarum (LMG P-20606), Lactobacillus paracasei (LMG P-26118), Pediococcus pentosaceus (LMG P-20608). Preferably, the composition also comprises Leuconostoc mesenteroides (LMG P-20607) and/or Bifidobacterium breve (LMG-P-26117).

A further composition that is specifically suitable for the treatment, amelioration or reduction of symptoms of IBD comprises Lactobacillus plantarum (LMG P-20606); Lactobacillus paracasei (LMG P-17806); Pediococcus pentosacceus (LMG P-20608); Leuconostoc mesenterioides (LMG P-20607), and Bifidobacterium breve (LMG P-26117).

A further composition that is specifically suitable for the treatment, amelioration or reduction of symptoms of IBD comprises Lactobacillus plantarum (LMG P-20606); Lactobacillus paracasei (LMG P-26118); Pediococcus pentosacceus (LMG P-20608); Leuconostoc mesenterioides (LMG P-20607); and Bifidobacterium breve (LMG P-26117).

A further composition that is specifically suitable for the treatment, amelioration or reduction of symptoms of IBD comprises Lactobacillus plantarum (LMG P-20606) and Lactobacillus paracasei (LMG P-17806).

A further composition that is specifically suitable for the treatment, amelioration or reduction of symptoms of IBD comprises Lactobacillus plantarum (LMG P-20606) and Bifidobacterium breve (LMG P-26117).

Compositions that are specifically suitable for the treatment, amelioration or reduction of symptoms of IBD are Composition Nos. 19, 22, 37, 40, 47, 50 and 56 in Table 1.

Examples of areas where the microbiota has been shown to play an important role is on various immune functions including inflammation and on the gut-brain axis. The gut-brain axis is the bidirectional interaction between the gut and the central nervus system. Molecules of importance for this interaction are e.g. short chain fatty acids (SCFAs) which can be produced by the gut microbiota if a suitable substrate is available e.g. soluble fibres or oligosaccharides. The most abundant short chain fatty acids (SCFAs) in stool and body fluids are formic acid, acetic acid, propionic acid and butyric acid. SCFAs are multifunctional molecules, being e.g. an essential energy source for local intestinal cells, but also influencing barrier function, neurotransmitter release, microglial maturation and activation, neural proliferation, mitochondrial function, oxidative stress, immune-modulation, and anti-inflammatory processes. SCFAs can regulate brain function and behavior via a number of potential mechanisms and dysregulation of these pathways may contribute to autism and related disorders. Butyric acid, formic acid, acetic acid and propionic acid have previously been reported to have anti-inflammatory effects. SCFAs regulate several leukocyte functions including production of cytokines eicosanoids and chemokines. The ability of leukocytes to migrate to the foci of inflammation and to destroy microbial pathogens also seems to be affected by the SCFAs. Studies have shown that SCFA at physiological concentration may affect the pathogenesis of some viruses e.g. Herpesvirus at the epithelial site. The use of the SCFA butyrate or microbial metabolism for production of butyric acid has been speculated to be one way to support the treatment of SARS-CoV2-infection.

VCAM-1 (Vascular Cell Adhesion Molecule) is a receptor used by some types of Adenovirus during infection. VCAM-1 is a cell surface glycoprotein expressed on e.g. endothelial cells and immune cells such as macrophages and dendritic cells. VCAM-1 was originally identified as a cell adhesion molecule that helps to regulate inflammation-associated vascular adhesion and the transendothelial migration of leukocytes, such as macrophages and T cells. Recent evidence suggests that VCAM-1 is closely associated with the progression of various immunological disorders.

ICAM-1 (InterCellular Adhesion Molecule) is a receptor used by most Rhinoviruses and some Coxsackievirus as a receptor during infection. ICAM-1 is also important for the infection cycle of influenzavirus. ICAM-1 (also known as CD54 (Cluster of Difference 54)) is a cell surface glycoprotein that promotes signalling in between cells related to the immune response. ICAM-1 is expressed on endothelial cells, immune cells and on respiratory endothelium where it can serve as a receptor for viruses, e.g. human rhinovirus (HRV). Furthermore, patients suffering from a severe form of Covid-19 (caused by the coronavirus SARS-CoV-2) have markedly increased levels of ICAM-1. ICAM-1 is a receptor for 90% of all Rhinovirus. Studies have shown that Rhinovirus and some types of Adenovirus and Coxsackivirus share receptors. ICAM-1 has also been demonstrated to regulate the survival of influenzavirus in lung epithelial cells during the early stages of infection. Thus, reducing the expression of ICAM-1 is anticipated to have an effect on the clinical manifestation of virus infections, especially infections of the upper respiratory tract.

IL-12/IL23p40 has been identified as important in Inflammatory Bowel Disease (IBD), i.e. in ulcerative colitis and Crohn's disease, and is a target for medical treatment of the disease. Thus, reducing the expression of IL-12/IL-23p40 is anticipated to have an effect on the clinical manifestation of this disease.

In the experiments described below, the effect of a composition (corresponding to Composition No. 4) on the levels of i.a. IL-12/IL-23p40, ICAM-1, and SCFAs has been studied.

### Experiments and Studies

Immunological Markers and Short Chain Fatty Acids in Healthy Individuals and Individuals with Inflammation

The levels of selected immunological markers and short chain fatty acids were measured in blood samples from healthy individuals (n=57) and individuals diagnosed with inflammatory disorder/inflammatory load/inflammatory burden (n=154, 8-55 years old, both men and women). A variety of immune activity markers of which some also are virus receptors were analysed.

As can be seen in Table 2, individuals diagnosed with inflammatory disorder/inflammatory load/inflammatory burden have significantly (p<0.005) lower expression of IL-10 (an anti-inflammatory marker) and significantly (p<0.005) higher expression of the inflammatory markers and upper tract respiratory disease virus receptors sICAM-1 and sVCAM-1.

This clearly demonstrates that individuals with inflammatory disorder/inflammatory load/inflammatory burden express an inflammatory pattern of immune markers and express virus receptors at higher levels than healthy individuals.

TABLE 2
Inflammatory activity markers/virus receptors, expressed
in healthy individuals (“Control group”) and
individuals diagnosed with inflammatory disorder/inflammatory
load/inflammatory burden (“Inflammatory group”)

| | | Inflammatory | Significance, |
|---|---|---|---|
| | | group at | difference between |
| | Control group | baseline | control group and |
| Marker/ | Amount pg/ml | Amount pg/ml | inflammatory group |
| receptor | (median) | (median) | (p) |
| IL-10 | 0.60 | 0.25 | 0.00202 |
| sICAM-1 | 400,000 | 500,000 | 0.00202 |
| sVCAM-1 | 400,000 | 480,000 | 0.00003 |

Formic and propionic acids (short chain fatty acids) were lower in the individuals with inflammatory disorder/inflammatory load/inflammatory burden compared to the control group of 57 healthy individuals. Short chain fatty acids in plasma correlated positively with IL-10, i.e. individuals with a low level of short chain fatty acids also had a low level of the anti-inflammatory cytokine IL-10. This was seen both in children and in adults (data not shown).

Influence of a Synbiotic Composition According to the Present Disclosure on Inflammatory Markers and Virus Receptors in Individuals with Inflammation

A human intervention study was performed in a group of individuals (n=49, children, both boys and girls) diagnosed with inflammatory disorder/inflammatory load/inflammatory burden. The effect of a synbiotic composition according to the present disclosure on plasma levels of immune markers, virus receptors and short fatty acids in children diagnosed with inflammatory disorder/inflammatory load/inflammatory burden was investigated. The synbiotic composition included Lactobacillus plantarum LMG P-20606, Lactobacillus paracasei LMG P-17806, Pediococcus pentosaceus LMG P-20608 and the fibres inulin, pectin, resistant starch and beta-glucan. The total amount of bacteria was 4×10¹¹ and the total amount of fibres was 10 g. The individuals diagnosed with inflammatory disorder/inflammatory load/inflammatory burden were randomized into one group (n=28) receiving the synbiotic composition according to the present disclosure and one group (n=21) receiving a maltodextrin placebo. The synbiotic composition according to the present disclosure was consumed once daily for nine weeks. Immune markers and virus receptors were analysed in plasma at start of the study (T=0) and after nine weeks of intervention (T=9).

After 9 weeks, children receiving the synbiotic composition according to the present disclosure had a significantly (p<0.05) reduced level of the inflammatory marker IL-12/IL-23p40, the upper tract respiratory disease virus receptor sICAM and of transforming growth factor β3 (TGF-β3) in plasma compared to the levels at start of the intervention study (Table 3).

The results clearly show that the synbiotic composition lowered the expression of IL-12/IL-23p40, sICAM and TGF-β3, which all are inflammatory marker. IL-12/IL-23p40 is a target for drugs against inflammatory diseases and ICAM also serves as a receptor for viruses. Thus, intake of the synbiotic composition according to the present disclosure lowers the level of an important receptor for viruses infecting the upper respiratory track, thus reducing the entry points of these viruses. Furthermore, the results show that the synbiotic composition lowers the expression of the pro-inflammatory molecules IL-12/IL-23p40, a target, the reduction of which is used in the treatment of ulcerative colitis and/or Crohn's disease. Thus, the synbiotic composition according to the present disclosure can be used in the alleviation or treatment of ulcerative colitis and/or Crohn's disease.

TABLE 3
Time effects over 9 weeks on inflammatory markers

| | T = 0 | | Significance |
|---|---|---|---|
| | Amount | T = 9 weeks | difference between |
| | pg/ml | Amount pg/ml | T = 0 and T = 9 weeks |
| Marker/receptor | (median) | (median) | (p) |
| IL-12/IL-23p40 | 260 | 238 | 0.0281 |
| sICAM-1 | 740,000 | 670,000 | 0.0242 |
| TGF-β3 | 4.1 | 3.6 | 0.0405 |

Influence of a Synbiotic Composition According to the Present Disclosure on Short Chain Fatty Acids and their Correlation to Inflammatory Markers and Virus Receptors

A human intervention study was performed in a group of individuals (n=49, children, both boys and girls) diagnosed with inflammatory disorder/inflammatory load/inflammatory burden as described above. The synbiotic composition increased the levels of propionic and acetic acid, which correlated negatively to the levels of sICAM-1 and sVCAM-1, i.e. the proinflammatory markers and receptors were high when the short chain fatty acid level was low (data not shown). Thus, treatment with the synbiotic composition according to the present disclosure is associated with lower vascular inflammation. The results clearly show that the level of short chain fatty acids is associated to the level of immune markers and receptors, and with low levels of short chain fatty acids associated to a pro-inflammatory pattern of the immune marker and an enhanced expression of the virus receptors. In the study it was also clear that the synbiotic composition increased the plasma level of short chain fatty acids which will stimulate a low inflammation.

### In Vivo IBD-Study

### Objective and Method

A study was conducted in mice with the objective to demonstrate the effect of test products (different pre- and probiotics compositions according to the present disclosure) on intestinal inflammation induced by dextran sodium sulfate (DSS).

This study followed principles of Good Laboratory Practice (GLP) as defined by the Organization for Economic Co-operation and Development (OECD) as applicable. Procedures involving the care and the use of animals in the study was reviewed by the University of Lund Ethics Review Committee on Animal Experiments. During the study, the care and use of animals was conducted in accordance with the principles outlined in the current Guide to the Care and Use of Experimental Animals.

The study was conducted by the Dept Biology, Lund University.

### Tested Products

Tested products were a combination of probiotic bacteria and prebiotic fibres:
Product 1 (corresponding to Composition No. 37 in Table 1; also referred to as SB1): Lactobacillus plantarum (LMG P-20606); Lactobacillus paracasei (LMG P-17806); Pediococcus pentosacceus (LMG P-20608); Leuconostoc mesenterioides (LMG P-20607); Bifidobacterium breve (LMG P-26117); β-glucan from oat; Inulin from chicory root; Pectin from citrus fruits; and Resistant starch from corn.
Product 2 (corresponding to Composition No. 56 in Table 1; also referred to as SB2): Lactobacillus plantarum (LMG P-20606); Bifidobacterium breve (LMG P-26117); and Resistant starch from corn.
Product 3 (corresponding to Composition No. 47 in Table 1; also referred to as SB3): Lactobacillus plantarum (LMG P-20606); Lactobacillus paracasei (LMG P-26118); and Resistant starch from corn.
Product 4 (corresponding to Composition No. 50 in Table 1; also referred to as SB4): Lactobacillus plantarum (LMG P-20606); Pediococcus pentosacceus (LMG P-20608); and Resistant starch from corn.

### Control Groups

Three control groups were run in parallel, one which was only run in parallel to the experiment but where the mice were not treated with anything, one given only 1% DSS in drinking water and no pro/prebiotics and one that was given 1% DSS and conventional treatment with mesalamine.

### Animals Used in the Investigation:

In all the groups, the ones given probiotics (i.e. Product 1, 2, 3 and 4 corresponding to composition 37, 56, 47 or 50) and the three control groups, twelve mice were included in each group. For initial testing of colon sensitivity, two groups of mice, one given no treatment and one given 1% DSS for 4 days, were used before gut sensitivity testing.

The daily dose of bacteria to a mouse was 2×10E8 CFU. The daily dose of fiber to a mouse was 0.0025 g/day.

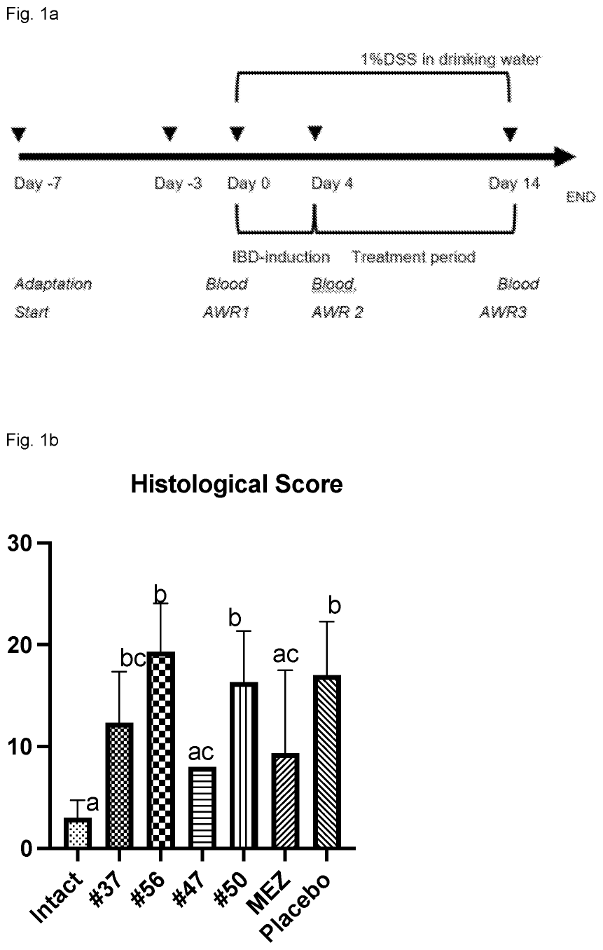

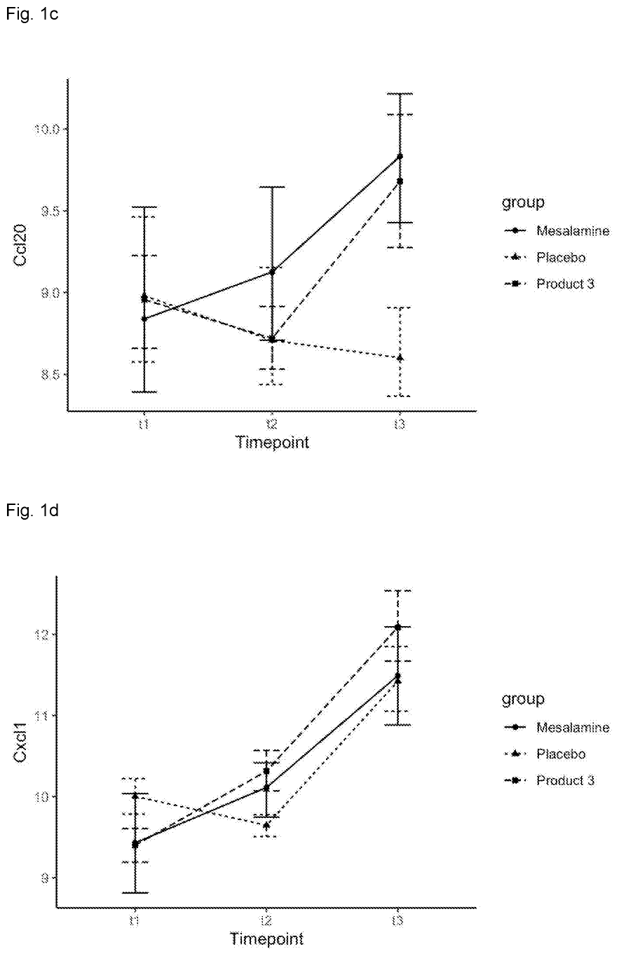

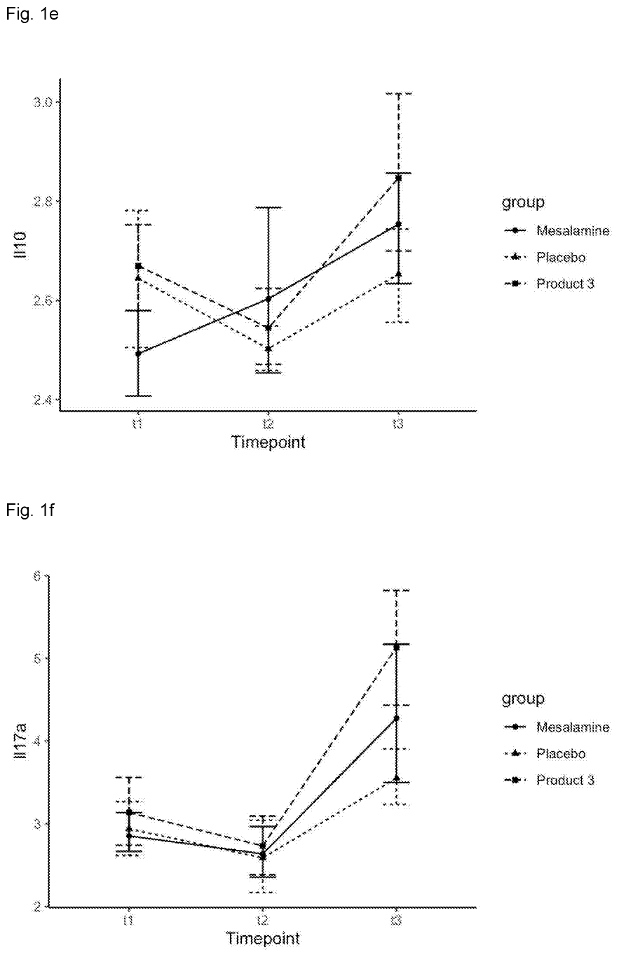

Female and male mice of the line C57BL were used. The age of the animals was 10 weeks. Average weight of the animals was 20-25 g at the start. The mice were fed a standard commercial diet (Lactamin, Vadstena, Sweden) which was available ad libitum as was water. Animal body weight and feed intake after adaptation period was measured daily, and water intake measured weekly. All animals but the control group, without any treatment, was given 1% DSS to induce an inflammation in the gut. A schematic overview of the study timetable, its design and animals' randomization is given in FIG. 1.

### Sampling

Blood samples were collected from the submandibular vein and isolated plasma was frozen at −80-C until analysis (plasma immunological profile).

Stool samples were collected both before starting the treatment as well as after DSS treatment and after product treatment. Stool samples were frozen until analysis of calprotectin using S100A8/A9 (MRP8/14, Calprotectin, Mouse/Rat) ELISA kit, KR6936, ImmunDiagnostik, Bensheim, Germany).

The study was finalized by all animals euthanized at the end of the study by treatment by an overdose of isoflurane/transcardial perfusion.

For post-mortem analysis and tissue sampling, half of the mice from each group was euthanized by isoflurane overdose. Spleen and colon were dissected from these mice and weighed and colon length was measured. Samples of large intestine (caecum, proximal and distal colon) were collected and fixed in 10% neutral formalin solution fixative for 24 hours for further morphological analysis.

The other half of the mice from each group was euthanized by transcardial perfusion with Thyrodes solution+LANA's Fixative. Caecum, proximal and distal colon was dissected from these mice. The intestinal samples were kept in the same fixative overnight and then transferred to 70% alcohol for further morphological analysis.

### Analysis

Mice colon hypersensitivity was assessed by measuring the threshold intensity of the abdominal withdrawal reflex (AWR) arising in response to colorectal distention. Distention was applied using an arterial embolectomy catheter (4F-Fogarty, Edwards Lifesciences LLC, Irvine, CA, USA) which was inserted rectally into the descending colon of anesthetized mice (2% isoflurane) and fixed at the base of the tail. AWR measurements were carried out 30 min after wake-up and reorientation of the animals. A visual observation of the reaction to the rapid phase stretching of the balloon for 20 s in ascending order (0.1, 0.25, 0.35, 0.5, and 0.65 ml) was carried out. The response of the animal to colorectal distention was assessed on an AWR scale: 0, no behavioral response; 1, brief movement of the head, followed by immobility; 2, contraction of abdominal muscles; 3, lifting the abdomen, and 4, body flexion and pelvic lift. The visceral pain threshold (VPT) was defined by the stimulus intensity whereby to evoke a visible contraction of the abdominal wall.

Morphological analysis was performed for three intestinal segments for six animals/group.

At dissection, samples were collected from the large intestine: caecum, proximal and distal colon, and fixed in 10% neutral formalin solution fixative for 24 hours. Next, tissue pieces were dehydrated and embedded in paraffin according to standard histological techniques. Paraffin-embedded tissues were sliced using rotor microtome into sections 4.5 microns (μm) thick. After overnight drying, slides were deparaffinized by incubation in xylene and decreasing concentrations of ethanol. Finally, standard hematoxylin and eosin staining was performed for morphology analysis.

Statistical analysis was performed as follows: The distribution of the parameters was checked using a Shapiro-Wilk normality test. One-way ANOVA or Kruskal-Wallis test corrected for multiple comparisons was used dependently on the data distribution. In all statistical analyses p s 0.05 was considered significant. All analyses were carried out using Prism, version 9.1.0 (GraphPad Software, Inc, San Diego, CA, USA).

### Analysis of Biological Markers in Plasma

Blood samples collected in EDTA tubes from the mice and plasma was collected. Selected biomarkers were analysed using Proximity Extension Assay (PEA) technology at Olink, Uppsala, Sweden. The technique combines immune technology with qPCR enabling a high-throughput, multiplex immunoassays of proteins using minimal volumes of serum, plasma, or almost any other type of biological sample. In total 75 different markers were analysed.

Plasma samples from all experimental groups were analysed with the PEA-technique.

### Results

To indicate colitis development and its severity in mice, clinical assessment of animals was performed daily. Clinical scores indicated the colitis development already at day 3 after 1% DSS introduction. Neither Mesalamine, which is approved by FDA for the treatment for IBD, nor the tested Products could influence body weight reduction. However, administration of Products 1, 2, and 3 led to significant (p<0,05) improvement in animals' wellbeing and general health status, including better stool consistency and faecal blood disappearance (Table 4a). The clinical score of mice from these groups (Products 1, 2, and 3) on the last day of study (Day 14) was significantly (p<0.05) lower than in the Placebo group and significantly lower than in the Mesalamine group i.e. when the mice were treated by a conventional treatment for IBD. Moreover, clinical score values observed in SB1 (Product 1) treated animals didn't differ from the those noted for healthy intact mice from Control group.

In conclusion: The Products 1, 2 and 3 (corresponding to Compositions No. 37, 56 and 47) led to a significant, 50-68%, improvement in health status in mice where IBD had been induced by 1% DSS (Table 4a). The effect seen from those three synbiotic products was also significantly better than when the conventional treatment with Mesalamine was used. This clearly indicates that compositions according to the present disclosure can be used in the prevention, amelioration, treatment, or reductions of symptoms in an individual suffering from IBD.

TABLE 4a
Clinical score general health on Day 14.

| | Body weight gain, | |
|---|---|---|
| | % of initial body | Clinical |
| Group | weight | Score |
| Control | 108.7 ± 6.3a | 0.0 ± 0.0a |
| Product 1 (corresponding to | 90.4 ± 8.8b | 2.4 ± 2.0ab |
| Composition No. 37) | | |
| Product 2 (corresponding to | 90.0 ± 9.5b | 3.5 ± 3.0b |
| Composition No. 56) | | |
| Product 3 (corresponding to | 90.2 ± 10.5b | 3.6 ± 2.9b |
| Composition No. 47) | | |
| Product 4 (corresponding to | 86.2 ± 10.0b | 6.0 ± 3.8bc |
| Composition No. 50) | | |
| MEZ | 88.0 ± 9.3b | 4.8 ± 2.1bc |
| Placebo | 90.8 ± 12.5b | 7.4 ± 1.2c |

Control: group of intact healthy mice (n = 12); Product 1-Product 4: animals receiving 1% DSS in drinking water and treated daily with respective products (Products 1-4) (n = 11-12); MEZ: animals receiving 1% DSS in drinking water and treated daily with Mesalamine suspension in olive oil (100 mg/kg body weight) (n = 11); Placebo: group receiving 1% DSS in drinking water and daily treated with water gavage as a vehicle (n = 12). Data were analysed using one-way ANOVA test (body weight gain) and Kruskal-Wallis test (clinical score) corrected for multiple comparisons and are presented as mean ± SD. Small letters describes significant differences within columns when p < 0.05.

The well-established marker for IBD and gut inflammation calprotectin was analysed in faeces after 1% DSS treatment showing that already after 4 days of treatment there was a significant 3-5 fold increase in the marker (Table 4b). The conventional treatment by mesalamine did not reduce the effect of DSS, but treatment with Products 1 and 2 prevented further increment of caprotectin levels and Product 3 significantly reduced (P<0.05) fecal calprotectin by 49%.

In conclusion: Products 1 and 2 (corresponding to Compositions No. 37 and 56, respectively) prevented increment in calprotectin and Product 3 (corresponding to Composition No. 47) significantly reduced fecal calprotectin with 49%. This clearly indicates that compositions according to the present disclosure can be used in the prevention, amelioration, treatment, or reductions of symptoms in an individual suffering from IBD.

TABLE 4b
Changes in faecal calprotectin levels.

| | Calprotectin, % | | |
|---|---|---|---|
| | Day −3 | Day 4 | Day 14 |
| Control | 100.00 ± 0.00a | 83.46 ± 48.03a | 109.26 ± 41.87a |
| Product 1 (corresponding | 100.00 ± 0.00a | 215.28 ± 48.44b | 202.75 ± 105.48ab |
| to Composition No. 37 | | | |
| Product 2 (corresponding | 100.00 ± 0.00a | 521.15 ± 323.22b | 257.46 ± 235.88ab |
| to Composition No. 56) | | | |
| Product 3 (corresponding | 100.00 ± 0.00a | 457.35 ± 261.72b | 283.60 ± 112.67c |
| to Composition No. 47) | | | |
| Product 4 (corresponding | 100.00 ± 0.00a | 260.97 ± 112.67b | 273.55 ± 189.38b |
| to Composition No. 50) | | | |
| MEZ | 100.00 ± 0.00a | 207.87 ± 51.58b | 429.60 ± 188.45c |
| Placebo | 100.00 ± 0.00a | 294.62 ± 137.95b | 377.54 ± 234.38b |

Control: group of intact healthy mice (n = 12); Product 1-Product 4: animals receiving 1% DSS in drinking water and treated daily with respective products (Products 1-4) (n = 11-12); MEZ: animals receiving 1% DSS in drinking water and treated daily with Mesalamine suspension in olive oil (100 mg/kg body weight) (n = 11); Placebo: group receiving 1% DSS in drinking water and daily treated with water gavage as a vehicle (n = 12). Data were analysed using one-way ANOVA test (body weight gain) and Kruskal-Wallis test (clinical score) corrected for multiple comparisons and are presented as mean ± SD. Small letters describes significant differences within columns when p < 0.05.

1% DSS-induced intestinal inflammation was characterized by inflammatory cell infiltration, abnormal crypt architecture, hyperplasia and edema. Some mild histological alterations were detected in the intact control group, however, all animals treated with DSS developed colitis. However, Mesalamine and Product 3 treatment significantly reduced inflammation, improved crypt structure and ameliorated edema by ca 50% (FIG. 1b).

In conclusion: Product 3 (corresponding to Composition No. 47) treatment significantly reduced inflammation, improved crypt structure and ameliorated edema by ca 50%. This clearly indicates that compositions according to the present disclosure can be used in the prevention, amelioration, treatment, or reductions of symptoms in an individual suffering from IBD.

The use of the synbiotic products significantly influenced several plasma biomarkers and in particular immunoactive markers. Three markers were influenced similarly independently of the product used. FIGS. 1c-1f show results for those markers, i.e. Ccl20, IL17a and Cxcl1, in plasma in the group of mice receiving product 3 (composition 47). The pattern of reaction is similar for these three markers when any of the four combination of the synbiotic composition was used i.e. the amount of the marker is relatively stable after DSS treatment (t=2) but increases significantly after treatment with the synbiotic composition (t=3), thus the figures are typical examples of what happens when the synbiotic products are given to mice after IBD induction. The increase in the amount of the markers is usually a little higher than the increase after mesalamine treatment. Mesalamine is an established way of treating IBD. For the anti-inflammatory cytokine IL10 the pattern is somewhat different i.e. the amount of this marker in plasma decreases significantly after treatment with DSS but increases again when the synbiotic product is given. This result on IL10 was only significant in the group receiving product 3. Other markers influenced by the different synbiotic compositions is different between the products meaning that the specific composition of a product is important for a specific effect (Table 4c).

TABLE 4c
Plasma markers where the change over time is different in Product compared to placebo, at T1 to
T2, T2 to T3, and T1 to T3. Markers with p-value below 0.05 in T2 to T3 or T1 to T3 are listed.

| | Placebo Mean (SD) | | | Product Mean (SD) | |
|---|---|---|---|---|---|
| Marker | T2 minus T1 | T3 minus T2 | T3 minus T1 | T2 minus T1 | T3 minus T2 |

Product 1

| | | | | | |
|---|---|---|---|---|---|
| Il17a | 0.3519 (0.5937) | −0.9644 (0.8592) | −0.6125 (0.7647) | 0.139 (0.5627) | −3.085 (2.1354) |
| Tnfrsf11b | 0.1277 (0.3085) | −0.0803 (0.2906) | 0.0473 (0.1606) | −0.05 (0.2705) | −0.713 (0.4571) |
| Ccl20 | 0.2731 (0.549) | 0.1061 (0.3342) | 0.3793 (0.5802) | 0.317 (0.7606) | −0.555 (0.8594) |
| VegId | −0.0589 (0.2476) | 0.1792 (0.2245) | 0.1203 (0.2777) | 0.05 (0.1368) | −0.032 (0.1507) |
| Lpl | −0.504 (0.3032) | −0.0582 (0.3419) | −0.5623 (0.5387) | −0.006 (0.4629) | −0.428 (0.3984) |
| Tpp1 | 0.0955 (0.2996) | 0.0681 (0.3335) | 0.1636 (0.3649) | −0.044 (0.2442) | −0.336 (0.4341) |
| Il17f | 0.093 (0.4158) | −0.4155 (0.6329) | −0.4186 (0.7575) | −0.035 (0.6452) | −1.473 (1.5854) |
| Ccl5 | −0.226 (0.2751) | −0.0106 (0.3212) | −0.2806 (0.2763) | 0.191 (0.2214) | 0.117 (0.3518) |
| Cxcl1 | 0.3554 (0.3142) | −1.7778 (0.5756) | −1.4225 (0.7439) | −0.185 (0.4764) | −2.477 (0.9796) |

Product 2

| | | | | | |
|---|---|---|---|---|---|
| Prdx5 | −0.0496 (0.4163) | −0.2095 (0.4065) | −0.2591 (0.5839) | −0.285 (0.5765) | −1.786 (1.4823) |
| Il17a | 0.3519 (0.5937) | −0.9644 (0.8592) | −0.6125 (0.7647) | 0.287 (0.2759) | −1.879 (0.7789) |
| Csf2 | −0.1004 (0.177) | −0.087 (0.2438) | −0.2187 (0.1565) | −0.009 (0.1596) | −0.409 (0.2972) |
| Ccl2 | 0.0429 (0.4363) | −0.5388 (0.3764) | −0.4959 (0.474) | −0.177 (0.9251) | −1.498 (1.0403) |
| Cxcl1 | 0.3554 (0.3142) | −1.7778 (0.5756) | −1.4225 (0.7439) | −0.591 (1.0402) | −2.715 (1.1859) |

Product 3

| | | | | | |
|---|---|---|---|---|---|
| Ccl20 | 0.2731 (0.549) | 0.1061 (0.3342) | 0.3793 (0.5802) | 0.236 (0.6359) | −0.959 (0.8818) |
| Cxcl1 | 0.3554 (0.3142) | −1.7778 (0.5756) | −1.4225 (0.7439) | −0.914 (0.5257) | −1.775 (0.8739) |
| Parp1 | 0.5088 (1.3727) | −0.0224 (1.8097) | 0.4864 (2.2125) | −0.853 (1.1549) | 1.611 (1.0905) |
| Pdgfb | −0.3271 (1.0878) | 0.5783 (1.9866) | 0.2512 (2.3147) | −1.705 (1.2842) | 2.583 (1.7054) |
| Ntf3 | −0.0557 (0.6429) | 0.2649 (0.7048) | 0.2191 (0.2477) | 0.229 (0.2534) | −0.241 (0.3478) |
| Il17a | 0.3519 (0.5937) | −0.9644 (0.8592) | −0.6125 (0.7647) | 0.404 (0.5028) | −2.401 (1.5702) |
| Cpe | 0.0646 (1.65) | 0.3941 (0.3813) | 0.4587 (0.3897) | 0.096 (0.2479) | 0.775 (0.3635) |
| Tgfb1 | −0.0922 (0.3459) | 0.144 (0.5013) | 0.0518 (0.5602) | −0.353 (0.3248) | 0.548 (0.3366) |
| Il10 | 0.202 (0.1156) | −0.1221 (0.2107) | −0.0676 (0.2792) | 0.129 (0.2199) | −0.374 (0.2464) |
| Tgfa | 0.2167 (0.3508) | 0.3138 (0.3719) | 0.5306 (0.3706) | 0.24 (0.4501) | 0.754 (0.5729) |

Product 4

| | | | | | |
|---|---|---|---|---|---|
| Ntf3 | −0.0557 (0.6429) | 0.2649 (0.7048) | 0.2191 (0.2477) | 0.302 (0.37) | −0.354 (0.2313) |
| Ccl20 | 0.2731 (0.549) | 0.1061 (0.3342) | 0.3793 (0.5802) | 1.243 (1.0697) | −0.889 (0.9684) |
| Il17a | 0.3519 (0.5937) | −0.9644 (0.8592) | −0.6125 (0.7647) | 0.028 (0.5497) | −2.353 (1.4588) |
| Ccl3 | −0.0815 (0.3562) | −0.2533 (0.9073) | −0.3348 (0.9737) | 0.04 (0.4079) | −1.117 (1.1079) |
| Tnfrsf11b | 0.1277 (0.3085) | −0.0803 (0.2906) | 0.0473 (0.1606) | 0.179 (0.28336) | −0.672 (0.5956) |
| Plxna4 | 0.0382 (0.5244) | 0.1329 (0.7193) | 0.2655 (0.6473) | 0.525 (1.1502) | −0.658 (0.4796) |
| Fst | −0.077 (0.3399) | 0.4464 (0.547) | 0.3693 (0.6359) | 0.23 (0.512) | 0.011 (0.5184) |
| Cxcl1 | 0.3554 (0.3142) | −1.7778 (0.5756) | −1.4225 (0.7439) | −0.369 (1.0303) | −2.567 (1.1638) |

| | | p-value_{Active} | | |
|---|---|---|---|---|
| | Product Mean (SD) | compared to Placebo | | |
| Marker | T3 minus T1 | T2 minus T1 | T3 minus T2 | T3 minus T1 |
| Product 1 | | | | |
| Il17a | −2.946 (1.9258) | 0.46 | 0.00032 | 0.0000914 |
| Tnfrsf11b | −0.763 (0.3544) | 0.237 | 0.00306 | 0.0000457 |
| Ccl20 | −0.238 (1.1855) | 0.897 | 0.0205 | 0.0205 |
| VegId | 0.018 (0.1601) | 0.173 | 0.0266 | 0.515 |
| Lpl | −0.434 (0.5149) | 0.0117 | 0.0676 | 0.696 |
| Tpp1 | −038 (0.413) | 0.237 | 0.0676 | 0.00439 |
| Il17f | −1.556 (1.4008) | 1 | 0.106 | 0.0426 |
| Ccl5 | 0.363 (0.2861) | 0.00967 | 0.272 | 0.00122 |
| Cxcl1 | −2.662 (1.2783) | 0.0117 | 0.122 | 0.0266 |
| Product 2 | | | | |
| Prdx5 | −1.989 (1.8422) | 0.44 | 0.0312 | 0.0727 |
| Il17a | −1.592 (0.8299) | 0.887 | 0.033 | 0.025 |
| Csf2 | −0.471 (0.461) | 0.57 | 0.042 | 0.776 |
| Ccl2 | −1.675 (1.484) | 0.962 | 0.0431 | 0.0878 |
| Cxcl1 | −3.305 (1.3832) | 0.161 | 0.193 | 0.00967 |
| Product 3 | | | | |
| Ccl20 | −0.723 (0.9357) | 0.809 | 0.00152 | 0.00619 |
| Cxcl1 | −2.69 (0.8623) | 0.0000113 | 0.918 | 0.00619 |
| Parp1 | 0.758 (0.3603) | 0.043 | 0.00795 | 0.973 |
| Pdgfb | 0.877 (2.081) | 0.0295 | 0.0101 | 0.282 |
| Ntf3 | −0.011 (0.2758) | 0.282 | 0.0125 | 0.0952 |
| Il17a | −1.996 (1.5433) | 0.654 | 0.0127 | 0.0127 |
| Cpe | 0.872 (0.4707) | 0.863 | 0.0295 | 0.0513 |
| Tgfb1 | 0.195 (0.3014) | 0.197 | 0.0357 | 0.605 |
| Il10 | −0.246 (0.2339) | 1 | 0.0379 | 0.272 |
| Tgfa | 0.994 (0.7126) | 0.809 | 0.043 | 0.043 |
| Product 4 | | | | |
| Ntf3 | −0.052 (0.327) | 0.0789 | 0.00276 | 0.0503 |
| Ccl20 | 0.355 (1.1177) | 0.0435 | 0.00762 | 0.905 |
| Il17a | −2.324 (1.2537) | 0.447 | 0.0101 | 0.0021 |
| Ccl3 | −1.077 (1.0533) | 0.78 | 0.0133 | 0.535 |
| Tnfrsf11b | −0.493 (0.5529) | 0.497 | 0.0172 | 0.00414 |
| Plxna4 | −0.133 (1.1186) | 0.536 | 0.0401 | 0.383 |
| Fst | 0.241 (0.329) | 0.315 | 0.0435 | 0.549 |
| Cxcl1 | −2.936 (0.6729) | 0.156 | 0.243 | 0.00065 |

Panel: Olink Mouse Exploratory panel consisting of 92 markers.
Values below lower detection limit of detection (LLOD) were excluded from analysis.
Marker with more than 30% of samples being below LLOD were removed.

In conclusion: The four synbiotic compositions influence three important immune markers in the same way or more as mesalamine, a substance established for the treatment of IBD. This indicates that the synbiotic compositions have a potential for treatment and/or alleviation of IBD. One of the synbiotic products (no 3 equivalent to composition 47) increased the amount of I110 in plasma. II 10 has important strong immune suppression properties which is important for treatment of inflammatory diseases such as IBD. This once again shows that the synbiotic composition can have an important role in treatment of IBD.

### User Survey 1

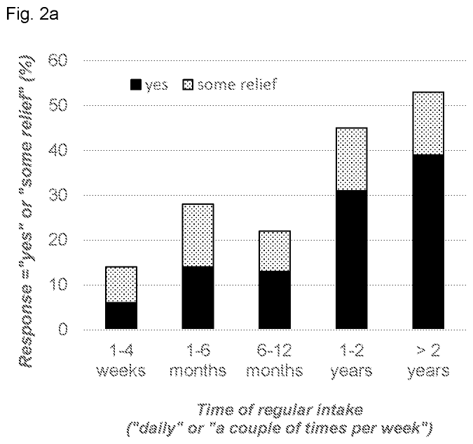

895 individuals (both men and women, from 18 years old) who had used a synbiotic composition according to the present disclosure (corresponding to Composition No. 19 in Table 1) for 1 week up to 2 years, sporadically up to every day, were questioned about their experiences and perceived effects of intake of the composition. The composition comprised Lactobacillus plantarum (LMG P-20606), Lactobacillus paracasei (LMG P-17806), Pediococcus pentosaceus (LMG P-20608), and Leuconostoc. mesenteroides (LMG P-20607) (total amount of of bacteria >1,5×10¹⁰ CFU/dose). Each dose further comprised inulin, pectin, resistant starch and betaglucan (total amount of fibers 3.75 g). Each individual independently reported any experienced positive effect(s) as well as any negative effects. None reported severe adverse effect or that medical treatment was needed. 504 individuals reported that one purpose of using synbiotic composition was “to strengthen the immune system”. 398 of these respondents had used the synbiotic composition daily or a couple of times per week. Out of these, 48 respondents had used the synbiotic composition 1-4 weeks. Responses to the question whether they felt that the synbiotic composition had helped them with problems with colds/seasonal flu are shown in Table 5a and in FIG. 2. In the same table, more details regarding time for use of the product and number of participants are shown.

TABLE 5a
Number and percentage of individuals who reported effects
of a synbiotic composition according to the present disclosure
(corresponding to Composition No. 19 in Table 1).

| | Do you feel that the |
|---|---|
| Time period the | synbiotic composition |
| synbiotic | helped you with |
| composition was | problems with |
| used a couple | colds/seasonal flu? |

| | | | |
|---|---|---|---|
| of times per | Number of | Yes, | Yes, I get |
| week or daily | individuals | absolutely | some relief |

| | | | | |
|---|---|---|---|---|
| 1-4 | weeks | 48 | 3 (6%) | 4 (8%) |
| 1-6 | months | 77 | 11 (14%) | 11 (14%) |
| 6-12 | months | 46 | 6(13%) | 4(9%) |
| 1-2 | years | 81 | 25 (31%) | 11 (14%) |
| >2 | years | 146 | 47 (39%) | 20 (14%) |

Notably, already after 1-6 months, 28% of the respondents reported that they had less problems with colds/seasonal flu. After 1-2 years of regular intake, 45% of the respondents reported that they had less problems and after more than 2 years of regular intake, 53% reported less problems with colds/seasonal flu. The group “6-12 months” included only 46 individuals, i.e. the smallest group in this survey and considerably smaller than the groups “1-2 years” and “more than 2 years”. Thus, in this group, a single individual's response will contribute to a larger extent to the percentage of reported effect of the synbiotic composition. However, the groups “1-2 years” and “more than 2 years” are large groups, with 81 and 146 individuals respectively, and the reported results from these groups must be considered as representative of a larger population. In conclusion: The results demonstrate that a synbiotic composition according to the present disclosure reduces problems with colds/seasonal flu.

### User Survey 2

93 individuals (both men and women, from 18 years old) who had used a synbiotic composition according to the present disclosure (corresponding to Composition No. 19 in Table 1) for 4 weeks, sporadically up to every day, were questioned about their experiences and perceived effects of intake of the composition on effects concerning the gut/IBD and on infection and inflammation. The composition comprised Lactobacillus plantarum (LMG P-20606), Lactobacillus paracasei (LMG P-17806), Pediococcus pentosaceus (LMG P-20608), and Leuconostoc. mesenteroides (LMG P-20607) (total amount of of bacteria >1,5×10¹⁰ CFU/dose). Each dose further comprised 3,75 g of fibers (inulin, pectine, resistant starch and beta-glucan). Each individual independently reported any experienced positive effect(s) as well as any negative effects. None reported severe adverse effect or that medical treatment was needed. g

### IBD

6 individuals reported that they suffered from IBD, e.g Crohn's disease, ulcerative colitis or other intestinal diseases. 4 of these respondents had used the synbiotic composition daily, almost every day or about half of the days. Responses to the question whether they felt that the synbiotic composition had helped them with problems related to the gastro intestinal tract (GI-tract) are shown in Table 5b.

As can be seen in Table 5b, already when the time period of regular intake of a synbiotic composition according to the present disclosure is as short as 4 weeks, the respondents report an improvement in symptoms related to IBD. In conclusion: the results indicate that a synbiotic composition according to the present disclosure may reduce problems with these symptoms and can be used in the treatment of IBD, including ulcerative colitis and Crohn's disease.

TABLE 5b
Number and percentage of individuals (out of 4 individuals)
who reported effects after 4 weeks of intake of a synbiotic
composition according to the present disclosure (corresponding
to Composition No. 19 in Table 1).

| | Do you feel that the synbiotic composition | | |
|---|---|---|---|
| | helped you with problems with [problem]? | | |
| | | | Total |
| | | | percentage that |
| | Yes, | Yes, I get | perceived an |
| Problem | absolutely | some relief | effect |
| General stomach-problems | 1 (25%) | 3 (75%) | 100% |
| Pain or discomfort in the | 1 (25%) | 2 (50%) | 75% |
| abdomen | | | |
| Constipation or diarrhea | 1 (25%) | 2 (50%) | 75% |
| Inflammation of the | 0 (0%) | 3 (75%) | 75% |
| intestine | | | |
| Flatulence/gas in the | 0 (0%) | 3 (75%) | 75% |
| stomach | | | |
| Often urgently need to get | 1 (25%) | 1 (25%) | 50% |
| to the toilet to empty the | | | |
| bowel | | | |
| Mucus or blood in the stool | 0 (0%) | 1 (25%) | 25% |

### Infections

76 of the 93 respondents had used the synbiotic composition daily, almost every day or about half of the days. Responses to the question whether they felt that the synbiotic composition had helped them with problems related to the immune system and infections are shown in Table 5c.

TABLE 5c
Number and percentage of individuals (out of 76 individuals)
who reported effects after 4 weeks of intake of a synbiotic
composition according to the present disclosure (corresponding
to Composition No. 19 in Table 1).

| | Do you feel that the synbiotic composition | | |
|---|---|---|---|
| | helped you with problems with [problem]? | | |
| | | | Total |
| | | | percentage that |
| | Yes, | Yes, I get | perceived an |
| Problem | absolutely | some relief | effect |
| Weak immune system | 3 (4%) | 19 (25%) | 29% |
| Recurrent bacterial | 5 (7%) | 18 (24%) | 31% |
| infections | | | |
| Recurrant fungal or viral | 5 (7%) | 16 (21%) | 28% |
| infections | | | |

As can be seen in Table 5c, already when the time period of regular intake of a synbiotic composition according to the present disclosure is as short as 4 weeks, respondents report an improvement in symptoms related to the immune system.

In conclusion: the results indicate that a synbiotic composition according to the present disclosure may reduce problems with these symptoms and can be used in the treatment and/or prevention of infections, such as viral infections.

Effect of a Composition According to the Present Disclosure on Persons with Symptoms Lasting Longer than 2 Months after Covid 19 Infection

### Participants

Otherwise healthy persons (male) who had experience symptoms of fatigue, smell or taste loss or other symptoms that could be related to Post Covid more than 2 month after a Covid 19 infection. The Covid 19 infection had been confirmed with PCR or antigen testing.

### Composition

The composition (corresponding to Composition No. 19 in Table 1) comprised Lactobacillus plantarum (LMG P-20606), Lactobacillus paracasei (LMG P-17806), Pediococcus pentosaceus (LMG P-20608), and Leuconostoc. mesenteroides (LMG P-20607) (total amount of bacteria was 4×10¹⁰ CFU/dose). Each dose further comprised inulin, pectin, resistant starch, betaglucan and rice fibers (total amount of fibers was 7,75 g). Dosage was one sachet of the product daily for 30 days. This is the ordinary daily recommended dosage of the product.

### Reporting

Participants were asked to daily (for 30 consecutive days during which they used the composition) answer questions regarding the intensity of symptoms related to post-COVID syndrome. The monitored symptoms were physical fatigue, mental fatigue, headaches, cough, heart palpitations, changes to sense of taste or smell. The level of symptoms were given as 1=no symptom, 2=some symptoms, 3=average level of symptoms, 4=quite a lot of symptoms, 5=severe symptoms. The participants were also asked if they were on sick leave due to post-COVID symptoms

### Results

Subject 1: Male, 43 years old, not on sick leave. Reported less physical and mental fatigue after three days of daily intake (from level 2 to level 1). Reported occurrence of headaches (level 3) on days 6, 17, 25 and 26 of the 30 days and heart palpitations (level 3 and 2) on 2 days. Notably, the subject report a decrease of pain in muscles and in joints (from level 5 to level 3; average level for days 1-10: 4.6; average level for days 11-20: 3.3; average level for days 21-30: 3.0).

Subject 2: Male, 53 years old, not on sick leave. Reported less mental fatigue after seven days of daily intake (from level 2 to level 1) with occurrence of mental fatigue on days 24-28. Reported less headaches after two days of daily intake (from level 2 to level 1). Reported an upset and tender stomach days 2 and 3.

Comments: These reports indicate that a composition according to the present disclosure may be used to treat, ameliorate or reduce symptoms associated with post-COVID syndrome.

1. A synbiotic composition for use in the treatment, prevention, amelioration, or reductions of symptoms in an individual suffering from Inflammatory Bowel Disease (IBD) or from an infection with a respiratory virus or from long-term effects after an infection with a respiratory virus, wherein the synbiotic composition comprises:
at least two bacterial strains chosen from the group consisting of:
Lactobacillus plantarum,
Lactobacillus paracasei,
Pediococcus pentosaceus,
Leuconostoc mesenteroides, and
Bifidobacterium breve;
and
at least one dietary fiber chosen from the group consisting of
inulin,
pectin,
beta-glucan,
resistant starch,
a galacto-oligosaccharide,
an isomalto-oligosaccharide, and
a rice fiber.

2. The synbiotic composition for use according to claim 1, wherein the Lactobacillus plantarum strain is Lactobacillus plantarum (LMG P-20606).

3. The synbiotic composition for use according to claim 1, wherein the Lactobacillus paracasei strain is chosen from the group consisting of
Lactobacillus paracasei (LMG P-17806), and
Lactobacillus paracasei (LMG P-26118).

4. The synbiotic composition for use according to claim 1, wherein
the Pediococcus pentosaceus strain is Pediococcus pentosaceus (LMG P-20608);
and/or the Leuconostoc mesenteroides strain is Leuconostoc mesenteroides (LMG P-20607);
and/or the Bifidobacterium breve strain is Bifidobacterium breve (LMG-P-26117).

5. The synbiotic composition for use according to claim 1, wherein the composition comprises at least one Lactobacillus plantarum strain, preferably Lactobacillus plantarum (LMG P-20606), and at least one Lactobacillus paracasei strain, preferably Lactobacillus paracasei (LMG P-17806) or Lactobacillus paracasei (LMG P-26118).

6. The synbiotic composition for use according to claim 5, wherein the composition further comprises at least one Pediococcus pentosaceus strain, preferably Pediococcus pentosaceus (LMG P-20608).

7. The synbiotic composition for use according to claim 5, wherein the composition further comprises at least one Leuconostoc mesenteroides strain, preferably Leuconostoc mesenteroides (LMG P-20607).

8. The synbiotic composition for use according to claim 5, wherein the composition further comprises at least one Bifidobacterium breve strain, preferably Bifidobacterium breve (LMG-P-26117).

9. The synbiotic composition for use according to claim 1, wherein the composition comprises Lactobacillus plantarum (LMG P-20606), Lactobacillus paracasei (LMG P-17806) or Lactobacillus paracasei (LMG P-26118), and Pediococcus pentosaceus (LMG P-20608).

10. The synbiotic composition for use according to claim 1, wherein the composition comprises inulin, pectin, beta-glucan and/or resistant starch.

11. The synbiotic composition for use according to claim 1, wherein the composition comprises resistant starch, a galacto-oligosaccharide, and/or an isomalto-oligosaccharide.

12. The synbiotic composition for use according to claim 1, wherein the total amount of bacteria in one dose is 1×10⁶ to 1×10¹³, such as 5×10⁶ to 1×10¹³, such as 1×10⁷ to 1×10¹³, such as 5×10⁷ to 1×10¹³, such as 1×10⁸ to 1×10¹³, such as 5×10⁸ to 5×10¹², especially 1×10⁹ to 1×10¹², such as 5×10⁹ to 9×10¹¹, such as 1×10¹⁰ to 8×10¹¹, such as 5×10¹⁰ to 7×10¹¹, such as 1×10¹¹ to 6×10¹¹, such as 3×10¹¹ to 5×10¹¹, such as 4×10¹¹ CFUs.

13. The synbiotic composition for use according to claim 1, wherein the total amount of fibers in one dose is between 0.1 to 20 g, such as 0.5 to 20 g, such as 1 to 20 g, such as 2 to 15 g, such as 2.5 to 12.5 g, such as 5 to 10 g, such as 6 to 8.5 g, such as 7.5 g.

14. The synbiotic composition for use according to claim 1, wherein the respiratory virus is chosen from the group consisting of:
adenovirus,
rhinovirus,
coxsackievirus,
coronavirus, and
influenzavirus.

15. The synbiotic composition for use according to claim 14, wherein the coronavirus is chosen from the group consisting of SARS-CoV, MERS-CoV and SARS-CoV2.

16. The synbiotic composition for use according to claim 1, wherein the long-term effects after an infection with a respiratory virus are long-term effects after a COVID-19-infection.

17. The synbiotic composition for use according to claim 1, wherein the Inflammatory Bowel Disease (IBD) is ulcerative colitis or Crohn's disease.

18. The synbiotic composition for use according to claim 1, wherein the synbiotic composition is administered orally or rectally or by tube feeding.

19. The synbiotic composition for use according to claim 1, wherein the synbiotic composition is administered as one dose 1 to 3 times a day, 1 to 7 times a week.

20. The synbiotic composition for use according to claim 1, wherein the synbiotic composition is in the form of a powder, a capsule, a tablet, a lozenge, a liquid, an emulsion, an enema, a suppository, or a tube feeding.

21. The synbiotic composition for use according to claim 1, wherein the synbiotic composition is a food supplement, a food product, a nutritional supplement, a natural remedy or a pharmaceutical product.
